(19) United States
(12) Patent Application Publication
(10) Pub. No.: US 2026/0165795 A1
(43) Pub. Date: Jun. 18, 2026

(54) INSTRUMENT STATE DETECTION FOR ROBOTIC MEDICAL SYSTEMS

(71) Applicant: Intuitive Surgical Operations, Inc., Sunnyvale, CA (US)

(72) Inventors: Pratibha Pandhare, Newark, CA (US); Saleh Tabandeh, San Carlos, CA (US)

(73) Assignee: Intuitive Surgical Operations, Inc., Sunnyvale, CA (US)

(21) Appl. No.: 18/984,545

(22) Filed: Dec. 17, 2024

Related U.S. Application Data
(60) Provisional application No. 63/611,509, filed on Dec. 18, 2023.

(51) Int. Cl. A61B 34/00; A61B 1/00; A61B 1/04; A61B 34/20; A61B 34/30

(52) U.S. Cl. CPC A61B 34/25; A61B 1/000096; A61B 1/00149; A61B 1/00154; A61B 1/0016; A61B 1/00193; A61B 1/04; A61B 2034/2055; A61B 2034/2065; A61B 2034/302

(57) ABSTRACT

Instrument state detection for robotic medical systems is described. A system can include one or more processors couple with memory. The one or more processors can receive one or more image frames captured via a camera for a medical session with a robotic medical system. The one or more processors can determine, based at least in part on the one or more image frames, a visual geometry of an instrument configured to perform a procedure via the robotic medical system during at least a portion of the medical session. The one or more processors can determine, based at least in part on data received from a sensor of a motor of the robotic medical system, a sensed geometry of the instrument. The one or more processors can provide, based at least in part on a comparison of the visual geometry with the sensed geometry, a notification of a state of the instrument during the medical session.

### CROSS-REFERENCE TO RELATED PATENT APPLICATION

This application claims, under 35 U.S.C. § 119, the benefit of, and priority to, U.S. Provisional Patent Application No. 63/611,509 filed December 18, 2023, the entirety of which is incorporated by reference herein.

### BACKGROUND

A robotic medical system can include an instrument for performing a medical session or procedure. For example, the instrument can be used to perform surgery, therapy, or a medical evaluation. The robotic medical system can articulate the instrument to perform the medical session or procedure.

### SUMMARY

Technical solutions disclosed herein can include determining instrument integrity using machine learning, image processing, and system algorithms. For example, a system can determine a visual geometry (e.g., position or orientation) of the tip of the instrument using video stream data received from a camera of an endoscope and visual processing. The system can determine a sensed geometry (e.g., position or orientation) of the tip of the instrument by using sensor data of a motor of the instrument (e.g., motor encoder data). The system can compare the visual geometry with the sensed geometry. The system can determine a state of the instrument or the tip of the instrument based on a deviation between the visual geometry and the sensed geometry. If the system detects a deviation or difference greater than a threshold amount between the sensed geometry and the visual geometry, the system can detect an error or fault condition indicating that the tip is damaged or was not properly installed on the instrument. The system can perform the state detection before a medical procedure is performed (pre-operatively), during a medical procedure (intraoperatively), or after the procedure is performed (post-operatively).

At least one aspect of the present disclosure is directed to a system including one or more processors coupled with memory. The one or more processors can receive one or more image frames captured via a camera for a medical session with a robotic medical system. The one or more processors can determine, based at least in part on the one or more image frames, a visual geometry of an instrument configured to perform a procedure via the robotic medical system during at least a portion of the medical session. The one or more processors can determine, based at least in part on data received from a sensor of a motor of the robotic medical system, a sensed geometry of the instrument. The one or more processors can provide, based at least in part on a comparison of the visual geometry with the sensed geometry, a notification of a state of the instrument during the medical session.

The one or more processors can operate a motor of a stereoscope of the including the camera to pass through a cannula. The one or more processors can operate the motor of the stereoscope to move the camera away from a longitudinal axis of the cannula by an amount. The one or more processors can detect that the camera is moved away from the longitudinal axis of the cannula by the amount. The one or more processors can operate at least one motor of an arm including the instrument to pass through the cannula responsive to a detection that that the camera is moved away from the longitudinal axis of the cannula by the amount.

The one or more processors can receive an indication that the instrument is installed on an arm of the robotic medical system. The one or more processors can compare the visual geometry with the sensed geometry to determine the state of the instrument responsive to the indication that the instrument is installed on the arm of the robotic medical system.

The one or more processors can detect that an event of the medical session occurred. The one or more processors can determine the visual geometry responsive to a detection of the event. The one or more processors can determine the sensed geometry responsive to the detection of the event. The one or more processors can compare the visual geometry with the sensed geometry to determine the state responsive to the detection of the event.

The one or more processors can determine a position or orientation of the camera of an endoscope of the robotic medical system. The one or more processors can determine the visual geometry of the instrument relative to the position or the orientation of the endoscope. The one or more processors can determine the sensed geometry of the instrument relative to the position or the orientation of the camera on the endoscope.

The one or more processors can detect, based at least in part on the one or more image frames, an object blocking a view of the instrument. The one or more processors can suppress the notification of an error state of the instrument responsive to a detection of the object blocking the view of the instrument.

The one or more processors can detect, based at least in part on the one or more image frames, a pattern of a sleeve covering at least a portion of an arm of the robotic medical system including the camera. The one or ore processors can generate the notification of the state of the instrument based on the pattern of the sleeve.

The one or more processors can detect, based at least in part on the one or more image frames, a marker of the instrument, the marker applied to the instrument. The one or more processors can track, based on the one or more image frames, the marker to determine the visual geometry of the instrument.

The one or more processors can periodically or continuously determine the visual geometry, determine the sensed geometry, and provide the notification of the state of the instrument during the medical session.

The one or more processors can execute a model trained on machine learning based on the data from the sensor of the motor and the one or more image frames to determine the state of the instrument. The one or more processors can provide the notification of the state of the instrument based on execution of the model.

The one or more processors can determine, based at least in part on the comparison of the visual geometry with the sensed geometry, that the instrument decoupled from an arm of the robotic medical system. The one or more processors can determine a time at which the instrument decoupled from the arm of the robotic medical system. The one or more processors can select at least one frame of a set of frames captured by the camera based on the time at which the instrument decoupled from the arm. The one or more processors can provide a graphical user interface including an indication that the instrument decoupled from the arm and the selected frame.

At least one aspect of the present disclosure is directed to a method. The method can include receiving, by one or more processors, coupled with memory, one or more image frames captured via a camera for a medical session with a robotic medical system. The method can include determining, by the one or more processors, coupled with the memory, based at least in part on the one or more image frames, a visual geometry of an instrument configured to perform a procedure via the robotic medical system during at least a portion of the medical session. The method can include determining, by the one or more processors, coupled with the memory, based at least in part on data received from a sensor of a motor of the robotic medical system, a sensed geometry of the instrument. The method can include providing, by the one or more processors, coupled with the memory, based at least in part on a comparison of the visual geometry with the sensed geometry, a notification of a state of the instrument during the medical session.

The method can include operating, by the one or more processors, coupled with the memory, a motor of a stereoscope of the including the camera to pass through a cannula. The method can include operating, by the one or more processors, coupled with the memory, the motor of the stereoscope to move the camera away from a longitudinal axis of the cannula by an amount. The method can include detecting, by the one or more processors, coupled with the memory, that the camera is moved away from the longitudinal axis of the cannula by the amount. The method can include operating, by the one or more processors, coupled with the memory, at least one motor of an arm including the instrument to pass through the cannula responsive to a detection that that the camera is moved away from the longitudinal axis of the cannula by the amount.

The method can include receiving, by the one or more processors, coupled with the memory, an indication that the instrument is installed on an arm of the robotic medical system. The method can include comparing, by the one or more processors, coupled with the memory, the visual geometry with the sensed geometry to determine the state of the instrument responsive to the indication that the instrument is installed on the arm of the robotic medical system.

The method can include detecting, by the one or more processors, coupled with the memory, that an event of the medical session occurred. The method can include determining, by the one or more processors, coupled with the memory, the visual geometry responsive to a detection of the event. The method can include determining, by the one or more processors, coupled with the memory, the sensed geometry responsive to the detection of the event. The method can include comparing, by the one or more processors, coupled with the memory, the visual geometry with the sensed geometry to determine the state responsive to the detection of the event.

The method can include determining, by the one or more processors, coupled with the memory, a position or orientation of the camera of an endoscope of the robotic medical system. The method can include determining, by the one or more processors, coupled with the memory, the visual geometry of the instrument relative to the position or the orientation of the endoscope. The method can include determining, by the one or more processors, coupled with the memory, the sensed geometry of the instrument relative to the position or the orientation of the camera on the endoscope.

The method can include executing, by the one or more processors, coupled with the memory, a model trained on machine learning based on the data from the sensor of the motor and the one or more image frames to determine the state of the instrument. The method can include providing, by the one or more processors, coupled with the memory, the notification of the state of the instrument based on execution of the model.

The method can include determining, by the one or more processors, coupled with the memory, based at least in part on the comparison of the visual geometry with the sensed geometry, that the instrument decoupled from an arm of the robotic medical system. The method can include determining, by the one or more processors, coupled with the memory, a time at which the instrument decoupled from the arm of the robotic medical system. The method can include selecting, by the one or more processors, coupled with the memory, at least one frame of a set of frames captured by the camera based on the time at which the instrument decoupled from the arm. The method can include providing, by the one or more processors, coupled with the memory, a graphical user interface including an indication that the instrument decoupled from the arm and the selected frame.

At least one aspect of the present disclosure is directed to one or more storage media storing instructions thereon, that, when executed by one or more processors, cause the one or more processors to receive one or more image frames captured via a camera for a manufacturing session for an instrument. The one or more processors can determine, based at least in part on the one or more image frames, a visual geometry of the instrument manufactured to perform a medical procedure via a robotic medical system. The one or more processors can determine, based at least in part on data received from a sensor of a motor, a sensed geometry of the instrument. The one or more processors can provide, based at least in part on a comparison of the visual geometry with the sensed geometry, a notification of a state of the instrument

The instructions can cause the one or more processors to determine, based at least in part on the comparison of the visual geometry with the sensed geometry, that the instrument decoupled from an arm of the robotic medical system. The instructions can cause the one or more processors to determine a time at which the instrument decoupled from the arm of the robotic medical system. The instructions can cause the one or more processors to select at least one frame of a set of frames captured by the camera based on the time at which the instrument decoupled from the arm. The instructions can cause the one or more processors to provide a graphical user interface including an indication that the instrument decoupled from the arm and the selected frame.

These and other aspects and implementations are discussed in detail below. The foregoing information and the following detailed description include illustrative examples of various aspects and implementations, and provide an overview or framework for understanding the nature and character of the claimed aspects and implementations. The drawings provide illustration and a further understanding of the various aspects and implementations, and are incorporated in and constitute a part of this specification. The foregoing information and the following detailed description and drawings include illustrative examples and should not be considered as limiting.

### BRIEF DESCRIPTION OF THE DRAWINGS

The accompanying drawings are not intended to be drawn to scale. Like reference numbers and designations in the various drawings indicate like elements. For purposes of clarity, not every component may be labeled in every drawing. In the drawings:

FIG. 1 depicts an example computing system of a robotic medical system to detect a state of an instrument.

FIG. 2 depicts an example robotic medical system including an instrument and an example endoscope inserted through a cannula into a patient.

FIG. 3 depicts an example method of detecting a state of an instrument of a robotic medical system.

FIG. 4 depicts an example computing architecture of a data processing system.

### DETAILED DESCRIPTION

Following below are more detailed descriptions of various concepts related to, and implementations of, methods, apparatuses, and systems to instrument state detection for robotic medical systems. The various concepts introduced above and discussed in greater detail below may be implemented in any of numerous ways.

A robotic medical system can have an instrument. The instrument can be or include a robotic arm, a robotic appendage, a robotic snake, or any other controllable member that can be articulated by the robotic medical system. The robotic medical system can perform a medical session or medical procedure. For example, the robotic medical system can articulate the instrument to perform surgery, therapy, or a medical evaluation with the instrument. The instrument can be or include a tip or end. The tip or end can be installed with or to the instrument. The tip can be removable or a permanent component of the instrument or the robotic medical system. For example, the tip can be a scalpel, a scissors, a monopolar curved scissors (MCS), a cautery hook tip, a cautery spatula tip, a needle driver, forceps, a tooth retractor, a drill, or a clip applier.

Before or during a medical procedure, a shaft of the instrument can be dressed or disposed within a sleeve. With the instrument dressed in the sleeve, a removable tip can be installed on an end of the instrument. Furthermore, an endoscope can be installed, clutched, and then guided through a cannula. The cannula can be a port or hollow structure that can be inserted into a patient. The cannula can create an opening for instruments to be inserted into the patient. Furthermore, the instrument with the installed tip can be clutched and guided through the cannula into the patient. During the medical procedure, the instrument can be removed once or multiple times to change or replace the tip of the instrument with another tip, e.g., a scissors could be replaced with a scalpel, and then replaced with a spatula.

However, mechanical issues or contact with other objects can affect the performance of the instrument or cause the tip to malfunction, deform, at least partially detach or fall from the end of the instrument. For example, if a technician does not properly or completely mount, install, or couple the tip with the instrument, the instrument or tip can malfunction, or the tip can become at least partially disconnected from the instrument. A partially disconnected tip may be unable to perform aspects of the medical procedure in an efficient or effective manner, thereby reducing the efficiency or effectives of the medical procedure, introducing delays in the performance of the medical procedure or increasing resource utilization (e.g., additional instruments, energy, or time). Furthermore, the tip can become damaged or break, a shaft of the instrument can bend or become damaged, or the sleeve of the instrument can become damaged.

If the sleeve installed over the shaft of the instrument is not property rolled up when the tip is installed on the instrument, the sleeve can get caught in the shaft and the tip, making the tip installation weak and prone to malfunction. Furthermore, when the instrument is inserted through the cannula into the patient, the instrument can make contact with, or rub against, an inner surface of the cannula, an endoscope already inserted through the cannula, or another already instrument already inserted through the cannula into the patient. This contact can weaken the coupling between the instrument and the tip, causing the tip to malfunction responsive to the contact or malfunction later during a medical procedure. Furthermore, in some situations a technician may loosely mount the tip to the instrument, and therefore a collision or friction against the cannula or another object can cause the tip to malfunction or fall off. The instrument may also bend due to a collision between the instrument and the cannula if the instrument is guided through the cannula improperly. Furthermore, the instrument sleeve can rupture or break if the instrument comes into contact with a sharp object, such as another instrument tip.

Some systems may have challenges in determining that state of a tip of an instrument. For example, a system might use sensor measurements from encoders without using machine learning or analyzing the sensor data with other endoscope image data, and can have difficulty accurately detecting the state of the tip, create false positive errors, or have difficulty in detecting that a tip is becoming detached from an instrument. The tip can, in some cases, be susceptible to wear and tear, which can be caused by use or sterilization of the tips after medical sessions. Furthermore, sensing apparatus that might be added to an instrument to monitor the state or integrity of the tip can add mass or weight, causing more energy to be consumed to operate the instrument or prevent the instrument from being moved or navigated into narrow areas.

To solve these and other technical problems, technical solutions of this disclosure can include determining instrument integrity using machine learning, image processing, and system algorithms. For example, a system can determine a visual geometry (e.g., position or orientation) of the tip of the instrument using video stream data received from a camera of an endoscope and visual processing. The system can determine a sensed geometry (e.g., position or orientation) of the tip of the instrument by using sensor data of a motor of the instrument (e.g., motor encoder data). The system can compare the visual geometry with the sensed geometry. The system can determine a state of the instrument or the tip of the instrument based on a deviation between the visual geometry and the sensed geometry. If the system detects a deviation or difference greater than a threshold amount between the sensed geometry and the visual geometry, the system can detect an error or fault condition indicating that the tip is damaged or was not properly installed on the instrument. The system can perform the state detection before a medical procedure is performed (pre-operatively), during a medical procedure (intraoperatively), or after the procedure is performed (post-operatively).

The system can use depth sensing or depth mapping to prevent raising a false alarm. The system can use depth sensing or occlusion detection with the video stream to detect a complete or partial occlusion, e.g., an object between the camera of the endoscope and the tip of the instrument. Responsive to detecting an occlusion, the system can prevent or suppress alarms or faults from being raised, e.g., faults raised based on pattern matching performed between the sensed geometry and the visual geometry. Furthermore, the system can utilize depth sensor or depth mapping with the video stream to detect that the tip of the instrument is bent by an anatomical structure of a patient, and suppress any detected faults or alarms to prevent a false alarm from being raised.

The system can detect that the tip of the instrument detached, decoupled, or dropped from the instrument. The system can generate a notification or alarm to notify an operator or another system that the tip of the instrument dropped from the instrument. The system can detect or track the location of the tip. For example, the system can estimate or determine a trajectory of the tip using the video stream. The system can use the trajectory of the tip to determine the position of the tip of the instrument.

The system can generate or provide a notification of the state of the instrument (e.g., tip dropped). The technology can track the location or estimate the trajectory of the dropped tip, and provide an indication on a graphical user interface of where the dropped tip may be located. The system can generate a map with an indication of a location of the dropped tip, and provide the map for display via a graphical user interface. The map can be an anatomical map or display of a patient. The system can use the detected location to retrieve the tip of the instrument, e.g., operate another instrument or endoscope to retrieve the tip. In some implementations, the system can be implemented for manufacturing or a manufacturing session to detect manufacturing issues, defects, or errors in the manufacturing of an instrument or tip.

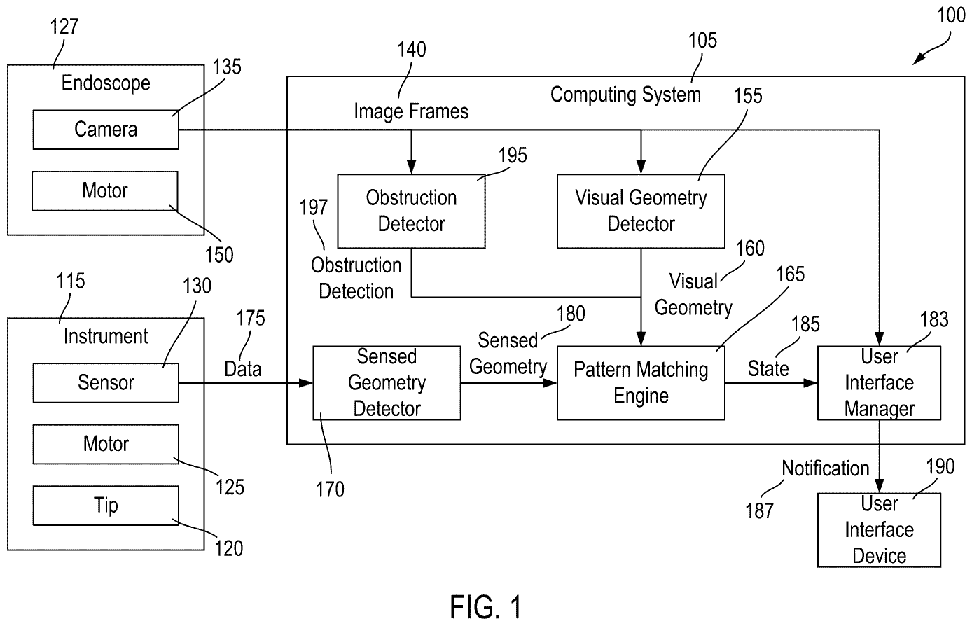

Referring now to FIG. 1, among others, an example computing system 115 of a robotic medical system 100 to detect a state of an instrument 115 is shown. The example computing system 105 can be a data processing system, a computer, a desktop computer, a control system, a console system, an embedded system, a cloud computing system, or any other type of computing system. The computing system 105 can be an on-premises system or an off-premises system. The computing system 105 can be a hybrid system, where some components of the computing system 105 are located on-premises, and some components of the computing system 105 are located off-premises.

The robotic medical system 100 can be a robotic system, a robotic apparatus, a robotic surgical system, a robotic therapy system, or a robotic treatment system. The robotic medical system 100 can be deployed or located at a hospital, a surgical room, a doctor's office, a military base, a military installation, an ambulance, a rotorcraft, or an aircraft. The robotic medical system 100 can be a single-port system or a multi-port system. The robotic medical system 100 can be a single-port system where a single-port or cannula provides entry into a patient, and multiple robotic instruments 115 or endoscopes 127 enter the patient via the single port. In a multi-port system, multiple ports or cannulas can provide entry for instruments 115 or endoscopes 127 into the patient. For example, only one instrument 115 or endoscope 127 can be inserted through each cannula. The instrument 115 can be installed with the robotic medical system 100 or with a patient side manipulator. The system 100 can be or include a manufacturing system to manufacture an instrument 115 or a tip 120. The system 100 can be a robotic manufacturing system, a computer numerical control (CNC) machine, a milling machine, an assembly machine, a press, etc.

The robotic medical system 100 can include at least one instrument 115. The instrument 115 can be or include a robotic arm, a robotic appendage, a robotic snake, or any other robotic member or component. The instrument 115 can include at least one tip 120. The tip 120 can be an instrument or apparatus, such as a scalpel, a scissors, a monopolar curved scissors (MCS), a cautery hook tip, a cautery spatula tip, a needle driver, a forceps, a tooth retractor, a drill, or a clip applier. The tip 120 can be coupled, attached, or connected with an end of the instrument 115. For example, the tip 120 can be coupled with an end of a shaft of the instrument 115. The tip 120 can be permanently installed with the instrument 115, e.g., permanently coupled with the shaft of the instrument 115 or integrally formed with the shaft of the instrument 115. The tip 120 can be removably coupled or removably installed with the instrument 115. For example, the tip 120 can be removed from the instrument 115 and replaced with a tip 120 of a different type before, during, or after a medical procedure.

The instrument 115 can include at least one motor 125. The motor 125 can be controlled by a technician, a doctor, or a surgeon via a control stick, joystick, keyboard, or input device. The motor 125 can be a brushed or brushless direct current (DC) motor, a stepper motor, or a servo motor. The computing system 105 can operate the motors 125 of the instrument 115 to articulate or control movement of the instrument 115. For example, the instrument 115 can move the tip 120 up and down along a z-axis, left and right about a y-axis, and back and forth about an x-axis. The instrument 115 can rotate the tip 120 about each of the x-axis, the y-axis, and the z-axis.

The medical computing system 105 can include an endoscope 127. The endoscope 127 can be or include a robotic arm, a robotic appendage, a robotic snake arm, or any other robotic member or component. The endoscope 127 can include at least one camera 135. The camera 135 can provide a video or image stream to the computing system 105. For example, the camera 135 can generate, capture, or create image frames 140. The camera 135 can transmit, provide, or stream image frames 140 to the computing system 105. The endoscope 127 can include at least one motor 150. The motor 150 can be controlled by a technician, a doctor, or a surgeon via a control stick, joystick, or input device. The motor 150 can be a brushed or brushless direct current (DC) motor, a stepper motor, or a servo motor. The computing system 105 can operate the motors 150 of the endoscope 127 to articulate or control movement of the endoscope 127. For example, the endoscope 127 can move the camera 135 up and down along a z-axis, left and right about a y-axis, and back and forth about an x-axis. The endoscope 127 can rotate the camera 135 about each of the x-axis, y-axis, and z-axis. The camera 135 can be a stereoscopic endoscope camera that captures stereoscopic image frames 140. The camera 135 can feed the stereoscopic images 140 into a pattern matching algorithm of a pattern matching engine 165 and depth sensing algorithm of an obstruction detector 195, or a visual geometry detector 155 during a medical procedure as well for continuous monitoring of the instrument 115 for detection of mechanical defects.

The computing system 105 can receive image frames 140 from the camera 135. The frames 140 can include images or pictures of the instrument 115 or the tip 120. The computing system 105 can process the image frames 140. The computing system 105 can include a visual geometry detector 155. The visual geometry detector 155 can detect a visual geometry 160 of the instrument 115 or the tip 120 of the instrument 115. For example, the visual geometry detector 155 can detect, based on the image frames 140, a current or average position or orientation of the tip 120. The visual geometry detector 155 can determine the position or orientation of the tip 120 relative to another object, point, axis, or plane. For example, the visual geometry detector 155 can determine the visual geometry 160 relative to a position or orientation of the endoscope 127 or the camera 135 of the endoscope 127. For example, the visual geometry detector 155 can determine a position or orientation of the camera 135 of the endoscope 127, and then determine the position or orientation of the tip 120 relative to the camera 135 of the endoscope 127.

The visual geometry detector 155 can detect the visual geometry 160 of the tip 120 before, during, or after a medical procedure or medical session. The visual geometry detector 155 can determine the visual geometry 160 periodically or continuously. The visual geometry detector 155 can provide the visual geometry 160 to a pattern matching engine 165. The visual geometry detector 155 can execute at least one model trained by machine learning to process the image frames 140. For example, the model trained by machine learning can be a neural network, such as a convolutional neural network or other image processing network. The visual geometry detector 155 can execute the model to detect the presence of the instrument 115 or the tip 120, and predict or determine the position or orientation of the instrument 115 or the tip 120.

The instrument 115 can include at least one sensor 130. The sensor 130 can be or include an encoder. The encoder can be a mechanical, optical, or magnetic encoder. The encoder can measure an absolute position of the motor 125, or a position of the motors 125 relative to a position that the motor 125 was at when started for a particular session or at a particular time. The sensor 130 can be or include a current sensor, such as an inductive coil. The sensor 130 can measure an amount of current sourced by the motor 125, and therefore indicate an amount of torque of the motor 125. The sensor 130 can provide measurements or data 175, such as digital or analog signals, data packets, or other information, to the computing system 105.

The computing system 105 can include at least one sensed geometry detector 170. The sensed geometry detector 170 can receive the data 175 from a sensor 130. The sensed geometry detector 170 can determine a sensed geometry 180 of the instrument 115 or the tip 120 of the instrument 115. The sensed geometry detector 170 can determine the orientation or position of the instrument 115 or the tip 120 relative to the same object, point, axis, or plane as the visual geometry detector 155 determines the visual geometry 160. For example, the sensed geometry detector 170 can determine the position or orientation of the instrument 115 or the tip 120 relative to a position of the camera 135 or the endoscope 127.

The sensed geometry detector 170 can detect the sensed geometry 180 of the tip 120 before, during, or after a medical procedure or medical session. The sensed geometry detector 170 can determine the sensed geometry 180 periodically or continuously. The sensed geometry detector 170 can provide the sensed geometry 180 to a pattern matching engine 165. In some implementations, the visual geometry detector 155 and the sensed geometry detector 170 can monitor the position or orientation of any object coupled with the instrument 115 past an end of a shaft of the instrument 115. The sensed geometry detector 170 can determine the sensed geometry 180 with a model trained by machine learning, such as a neural network. For example, the sensed geometry detector 170 can execute a machine learning model based on the data 175.

The computing system 105 can include at least one pattern matching engine 165. The pattern matching engine 165 can match, compare, or contrast the sensed geometry 180 with the visual geometry 160. The matching performed by the pattern matching engine 165 can be performed from a point of view of the camera 135. The pattern matching engine 165 can determine a deviation between the visual geometry 160 and the sensed geometry 180. For example, the pattern matching engine 165 can detect that an orientation of the visual geometry 160 deviates from an orientation of the sensed geometry 180. The pattern matching engine 165 can detect that a position of the visual geometry 160 deviates from a position of the sensed geometry 180. The pattern matching engine 165 can detect a deviation in each degree of freedom of the instrument 115 or the tip 120, e.g., a deviation for a pitch of the tip 120, a deviation for a roll of the tip 120, a deviation of the yaw of the tip 120, a deviation of an x-axis position of the tip 120, a deviation of a y-axis position of the tip 120, and a deviation of a z-axis position of the tip 120. The pattern matching engine 165 can determine a deviation to be a distance between a position of the sensed geometry 180 and a position of the visual geometry 160.

The pattern matching engine 165 can compare the deviations to at least one threshold. If one or more of the deviations are greater than a first threshold, then the pattern matching engine 165 can detect that the tip 120 or the instrument 115 is in a fault state or an error state 185. The fault state or error state can indicate that the tip 120 is not properly installed on the instrument 115, has become damaged, or is at risk of falling off or decoupling from the instrument 115. The pattern matching engine 165 can compare the deviation to a second threshold less than the first threshold. If the deviation is greater than the second threshold, but less than the first threshold, the pattern matching engine 165 can detect that tip 120 or the instrument 115 is in a state 185 that requires servicing, should be checked or reviewed by a technician, or that the performance of the tip 120 or the instrument 115 is deteriorating. If the deviation is less than the second threshold, the pattern matching engine 165 can detect that the instrument 115 or tip 120 is operating properly, is installed properly, or that there are no faults, errors or alarms for the instrument 112 or the tip 120. The pattern matching engine 165 can apply different thresholds for comparing orientations versus comparing positions. For example, the pattern matching engine 165 can raise alarms for smaller deviations position than in orientation.

The pattern matching engine 165 can compare the sensed geometry 180 with the visual geometry 160 responsive to an event or a detection. The visual geometry detector 155 can detect the visual geometry 160 responsive to the event or the detection. The sensed geometry detector 170 can detect the sensed geometry 180 responsive to the event or the detection. The event can be the tip 120 being installed on the instrument 115. For example, the computing system 105 can receive an indication via a user interface device 190 that the tip 120 has been installed on the instrument 115. For example, the computing system 105 can receive feedback data 175 via the sensor 130 indicating that the motor 125 is loaded, e.g., an increase in current sourced by the motor 125 to turn the motor a particular amount. Responsive to a detection that the tip 120 is installed or coupled with the instrument 115, the pattern matching engine 165 can compare the visual geometry 160 with the sensed geometry 180 to determine the state 185 of the instrument 115 responsive to the indication that the instrument 115 is installed on an arm of the robotic medical system 100.

The pattern matching engine 165, in some implementations, can execute one or more models trained by machine learning algorithms to detect the state 185. The models can be neural networks, convolutional neural networks, recurrent neural networks, image processing neural networks, etc. The models can be executed directly on the data 175 received from the sensor 130 and directly on the image frames 140. In some implementations, a model is trained by machine learning on a training dataset including the data 175, image frames 140, and states 185 for the model to output. The model can be executed by the pattern matching engine 165 based on the image frames 140 and the data 175 to output the state 185. In some implementations, the model of the pattern matching engine 165 can execute based on the obstruction detection 197, the visual geometry 160, and the sensed geometry 180 to output the state 185. In some implementations, the pattern matching engine 165 can perform pattern matching with the visual geometry 160 and the sensed geometry 180. In some implementations, the pattern matching engine 165 can combine the pattern matching with a model that executes directly on the data 175 and the image frames 140. The state 185 can indicate an instrument tip 120 breaking, becoming damaged, a tip 120 becoming decoupled from the instrument 115, a sleeve covering a shaft of the instrument 115 becoming damaged or ruptured, a shaft of the instrument 115 bending, etc.

For example, the instrument 115 can include an arm or shaft. During a medical procedure, the shaft can be covered by a sleeve, which can be rolled back for the tip 120 to be installed on the end of the shaft. However, if the sleeve tangles with the coupling between the tip 120 and the shaft of the instrument 115, the installation of the tip 120 may be weak, and the tip 120 may become uncoupled from the instrument 115 during a medical procedure. Therefore, the images 140 which can capture the instrument 115 from a point of view including the sleeve, the model can be executed to perform processing to detect whether a pattern of the sleeve indicates an improper installation of the sleeve or the tip 120, e.g., the sleeve is jammed between an end of the shaft and the tip 120. The user interface manager 183 can generate the notifications 187 based on the state 185 generated with the model that processes images 140 of the sleeve.

The model executed by the pattern matching engine 165 can be trained based on image frames 140. The model can be trained by a machine learning algorithm with images of the instrument 115 with the tip 120 installed, without the tip installed, and tags indicating whether each image depicts an instrument 115 with a tip 120 or without a tip 120. The model can further be trained with images of the tip 120 properly installed or improperly installed, and tags indicating whether each image depicts a tip 120 properly installed on the instrument 115 or improperly installed on the instrument 115. The model can be executed on image frames 140 collected from the camera 135 to detect whether the tip 120 is present on the end of the instrument 115 or is missing from the end of the instrument 115. The model can be executed by the pattern matching engine 165 to detect that a pattern indicating that the tip 120 is present or is missing. The pattern matching engine 165 can cause the state 185 to indicate that the instrument 115 has the tip 120 installed, or does not have the tip 120 installed, based on the execution of the model. The pattern matching engine 165 can cause the state 185 to indicate that the instrument 115 has a properly installed tip 120 or an improperly installed tip 120 based on the execution of the model.

Furthermore, the pattern matching engine 165 can receive current measurements from the tip 120, to track a torque of the motor 125 or a force applied to the tip 120. The pattern matching engine 165 can track the force applied to the tip 120, and detect the state 185 with the tracked force. For example, the pattern matching engine 165 can determine or store baseline amounts of torque to articulate the instrument 115 in free space. The pattern matching engine 165 can detect that an amount of torque needed to articulate the instrument 115 is greater than a threshold, and therefore determine that the instrument 115 or the tip 120 is in an error state or is encountering an error.

The pattern matching engine 165 can compare the sensed geometry 180 with the visual geometry 160 responsive to a medical event occurring during a medical session. For example, the computing system 105 can detect via the image frames 140, via the data 175, or via information provided by a user via the user interface device 190, that a medical event has occurred or will occur. For example, the medical event can be the instrument 115 or the tip 120 being inserted through a cannula into a cavity of a patient. The medical event can be biological matter being removed by the tip 120. The medical event can be a medical procedure being completed. Responsive to a detection of the medical event is completed, the sensed geometry detector 170 can determine the sensed geometry 180, the visual geometry detector 155 can determine the visual geometry 160, and the pattern matching engine 165 can determine the state 185.

The computing system 105 can include at least one obstruction detector 195. The obstruction detector 195 can detect whether the tip 120 or instrument 115 is obstructed, occluded, or partially occluded by another object in a field of view of the camera 135. For example, the obstruction detector 170 can detect whether the tip 120 is occluded or partially occluded by an object. The obstruction detector 170 can use a depth sensing model trained by machine learning to detect the occlusion. The obstruction detector 170 can execute the image frames 140 with the depth sensing model. With the depth analysis, the obstruction detector 195 can determine whether there is an object or biological matter between the tip 120 and the camera 135. Responsive to a detection 197 that the tip 120 is obstructed, the obstruction detector 195 can provide the obstruction detection 197 to the pattern matching engine 165. The pattern matching engine 165 can suppress the notification 187 of an error state 185 of the instrument 115 or the tip 120 responsive to a detection of the object blocking the view of the instrument 115 or the tip 120. The pattern matching engine 165 can stop or pause comparing the visual geometry 160 with the sensed geometry 180 responsive to the obstruction detection 197. The sensed geometry detector 170 can pause or stop generating the sensed geometry 180 responsive to the obstruction detection 197. The visual geometry detector 155 can pause or stop generating the visual geometry 160 responsive to the obstruction detection 197.

The computing system 105 can include at least one user interface manager 183. The user interface manager 183 can generate a notification 187 based on the state 185. The user interface manager 183 can analyze the state 185 and raise an alarm or push a notification to the user interface device 190 responsive to a detection that the state 185 indicates a fault, error, or indication that the tip 120 has detached or decoupled from the instrument 115. The user interface manager 183 can cause a graphical user interface to be displayed on the user interface device 190. The sensed geometry detector 170 can periodically or continuously determine the sensed geometry 180. The visual geometry detector 155 can periodically or continuously determine the visual geometry 160. The user interface manager 183 can periodically or continuously provide the notification of the state of the instrument during the medical session. The notification 187 provided by the user interface manager 183 can be provided based at least in part on a comparison of the visual geometry 160 with the sensed geometry, during, before, or after the medical procedure.

The user interface manager 183 can provide information to locate a tip 120 that decoupled or dropped from the instrument 115 to the user interface device 190. For example, the pattern matching engine 165 can detect, based on a comparison of the sensed geometry 180 with the visual geometry 160 deviating by a threshold amount, that the tip 120 has decoupled or dropped form the instrument 115. The pattern matching engine 165 can record a time or time stamp at which the tip 120 decoupled from the instrument 115. The user interface manager 183 can provide a state 185 for the tip 120 indicating that the tip 120 decoupled from the instrument 115 and provide a timestamp at which the tip 120 decoupled from the instrument 115. The user interface manager 183, responsive to receiving the state 185 indicating that the tip decoupled from the instrument 115, can select at least one image frame 140 received from the camera 135 based on the time at which the tip 120 decoupled from the instrument 115. The user interface manager 183 can select an image frame 140 with a timestamp that matches the time at which the tip 120 detached from the instrument 115. Furthermore, the user interface manager 183 can select neighboring video clips, e.g., particular amount (e.g., 30 seconds, 1 minute, etc.) of video recorded by the camera 135 before the tip 120 dropped from the instrument 115 and after the tip 120 dropped from the instrument 115. The user interface manager 183 can select a set or group of image frames 140 or video clips that have timestamps within a window of time centered on the time at which the tip 120 detached from the instrument 115. The selected frames or videos can be displayed in a graphical user interface on the user interface device 190 by the user interface manager 183.

The user interface manager 183 can cause a graphical user interface to display a notification 187 that the tip 120 has decoupled from the instrument 115 and a frame or set of frames 140 that depict the tip 120 falling from the instrument 115. The user interface manager 183 can provide a map, such as an anatomical map, that indicates a predicted or expected location that the tip 120 fell to or is currently located. The user interface manager 183 can analyze the image frames 140 and detect a trajectory or motion of the tip 120 through the frames 140, e.g., with a Kalman filter, kinematics equations, or other tracking analysis. The user interface manager 183 can predict a location of the tip 120 with the predicted trajectory or path that the tip 120 takes.

The graphical user interface can include a video streamed from the camera 135. For example, the interface can include a live or historical video captured form the camera 135 that includes a navigation element or bar for a user to scan or seek through. The videos or the navigation bar can be tagged or annotated with the states 185 detected for the tip 120 at various times. For example, segments of time or segments of the video can be tagged as in a normal operating state 185, a state indicating the tip 120 is malfunctioning, or a state indicating that the tip 120 has decoupled from the instrument 115. In this regard, a user can skip or move to certain segments of the video based on a user input selecting a particular state 185. Furthermore, the graphical user interface can display performance metrics, objective performance metrics (OPIs) or key performance indicators (KPIs) of the instrument 115 or the tip 120, such as operation duration, efficiency, kinematic motion, procedure outcome, etc.

The computing system 105 can perform at least one operation to mitigate a defect or error of the tip 120 or the instrument 115. For example, the computing system 105 can operate the motors 125 to prevent the tip 120 from falling off or decoupling from the instrument 115 or to mitigate or to reduce an error state 185. For example, if encoder data 175 or image frames 140 indicate that the tip 120 is gradually falling off the instrument 115, the computing system 105 can change the operation or control of the motors 125 to stop the tip 120 from decoupling from the instrument 115. For example, the computing system 105 can top or minimize rotation of the tip 120, and move the tip 120 out of the cannula to be serviced by a patient. The computing system 105 can perform a parallel check on tip function in order to prevent the tip 120 from falling off. In some implementations, in response to detecting that the tip 120 is falling off the instrument 115, and before the instrument 115 has been inserted into a patient, the computing system 105 can operate the motors 125 to shake, move or perform a micromotion to determine whether the tip 120 is likely to or will fall off or whether it has been installed correctly.

If the tip 120 falls from the instrument 115, the computing system 105 can detect the position of the tip 120 (e.g., via a prediction or via an image captured by the camera 135) and operate or navigation another instrument 115 with a tip 120 that can pick up the fallen tip 120, articulate the tip 120 to pick up the fallen tip 120, and remove the fallen tip 120 from the patient out through the cannula.

In addition to implementing the computing system 105 during, before, or after a medical session or procedure, the computing system 105 can be implemented for manufacturing, for a manufacturing apparatus or system, for a manufacturing procedure or process. The computing system 105 can detect defects in an instrument during a manufacturing session, which can refer to or include any part or whole of a manufacturing process during which an instrument is being manufactured. The computing system 105 can implement a manufacturing procedure to manufacture the instrument 1115 or the tip 120 to perform a medical procedure via the robotic medical system 100. The computing system 105 can implement the various techniques described herein to detect a manufacturing defect. For example, during manufacturing of the robotic medical system 100, the instrument 115, or the tip 120, the computing system 105 can analyze whether the tip 120 is properly installed on the instrument 115. For example, a stereoscopic camera can inspect coupling ends of a shaft of the instrument 115 and the tip 120 to determine whether the manufacturing has been performed properly. For example, the computing system 105 can detect a manufacturing defect in the tip 120 or the instrument 115. Furthermore, the techniques of the computing system 105 can be implemented to check whether cleaning is performed property or the instruments have deteriorated prior to be inserted into a patient via a cannula. Furthermore, the computing system 105 can be used to provide post-operative training, case reviews, or key performance indications based on various states 185 determined during a medical procedure or session.

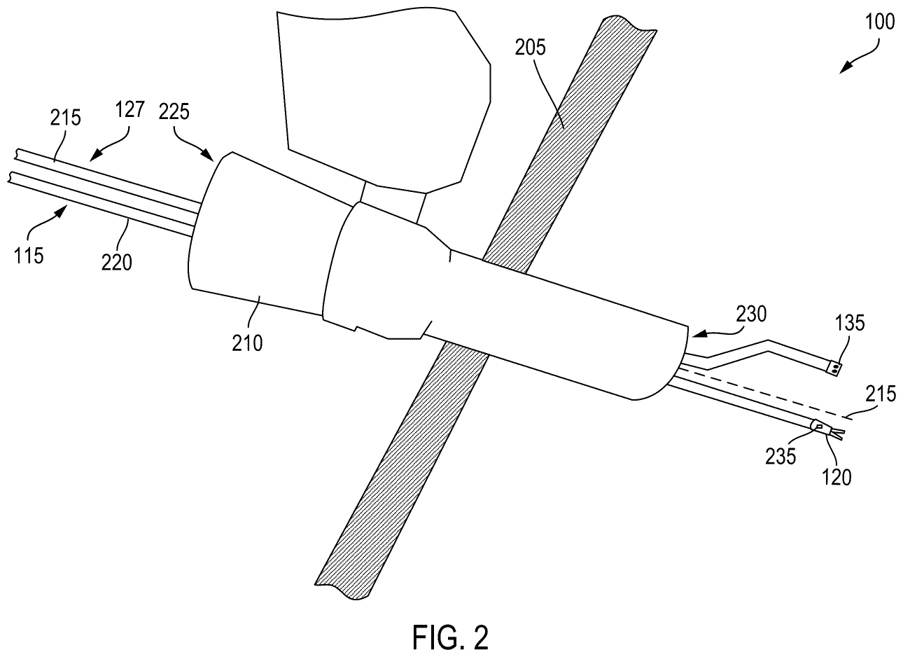

Referring now to FIG. 2, among others, a robotic medical system 100 including an instrument 115 and an endoscope 127 inserted through a cannula 210 into a patient is shown. The instrument 115 can include an arm, snake, or shaft 220. The shaft 220 can be a movable bendable structure that can move, bend, turn, or rotate via at least one motor 125. An end of the shaft 220 can be coupled with a tip 120. The tip 120 can be installed on the end of the shaft 220, e.g., snaped onto, screwed onto, etc. The tip 120 can fit over an end of the shaft 220. An inner surface of the tip 120 can be threaded, and can coupled with a threaded outer surface of an end of the shaft 220. In some implementations, the tip 120 can fit into a cavity on the end of the shaft 220. An outer surface of the tip 120 can be threaded, and can coupled with a threaded inner surface of a cavity on the end of the shaft 220.

The endoscope 127 can include an arm, snake, or shaft 215. The shaft 215 can be a movable bendable structure that can move, bend, turn, or rotate via at least one motor 150. An end of the shaft 215 can be coupled with the camera 135. The camera 135 can be installed on the end of the shaft 215, e.g., the camera 135 can be snaped onto the end of the shaft 215, the camera 135 can be screwed onto the end of the shaft 215, etc. The camera 135 can fit over an end of the shaft 215. An inner surface of the camera 135 can be threaded, and can coupled with a threaded outer surface of an end of the shaft 215. In some implementations, the camera 135 can fit into a cavity on the end of the shaft 215. An outer surface of the camera 135 can be threaded, and can coupled with a threaded inner surface of a cavity on the end of the shaft 215.

The robotic medical system 100 can include at least one cannula 210. The cannula can be a port, a tube, or a pipe. The cannula 210 can be inserted into a barrier 205, such as through skin, tissue, or an organ of a patient. The cannula 210 can hold an opening in the barrier 205 such that the instrument 115 and the endoscope 127 can be inserted through the barrier 205 into the patient. A first end or opening 225 of the cannula 210 can be positioned on a first side of the barrier 205 outside of the patient, while a second end or opening 230 of the cannula 210 can be disposed on a second side of the barrier 205 within a cavity of the patient. The endoscope 127 and the instrument 115 can be inserted through the cannula 210 into the first opening 225, through a channel of the cannula 210, and out of the second opening 230 into the cavity of the patient.

The computing system 105 can operate the motors 125 and 150 to avoid any collisions between the instrument 115 and the endoscope 127. The computing system 105 can operate the motors 150 of the endoscope 127 to insert the endoscope 127 into the cannula 210 and out through the opening 230. The computing system 105 can then operate the operate the motors 150 of the endoscope 127 to move the camera 135 away from a longitudinal axis 240 of the cannula 210. For example, the computing system 105 can move the camera 135 at least a predefined distance from the longitudinal axis 240. For example, the computing system 105 can move the camera 135 at least a distance away from the longitudinal axis 240 that the camera 135 is outside a boundary of the opening 230. The endoscope 127, when moving the camera 135 away from the longitudinal axis 240, can take on an s-shape, snake shape, or enter into a cobra pose.

Responsive to detecting that the camera 135 moving the predefined distance, the computing system 105 can determine to move the instrument 115 into the cavity of the patient. For example, responsive to determining that the camera 135 is moved by the predefined distance from the longitudinal axis 215, the computing system 105 can operate at least one motor 125 to cause the tip 120 to be guided into the opening 225 of the cannula 210, through the cannula, and out the exit 230 of the cannula. Because the camera 135 is in a cobra pose, and the camera 135 is moved away from the longitudinal axis 240, the likelihood that the tip 120 comes into contact with, bumps, or collides with the endoscope 127 and the camera 135 is decreased. In some implementations, the computing system 105 can automatically cause the endoscope 127 to peak when the tip 120 of the instrument 115 is outside a field of view of a camera 135 of the endoscope 127.

The instrument 115 or the tip 120 can include a marker 235. The marker 235 can be a visual identifier that the obstruction detector 195, the visual geometry detector 155, or the pattern matching engine 165 can utilize to detect and identify the tip 120, and track the orientation and position of the tip 120. For example, models executed by the computing system 105 can be trained based on images of the tip 120 including the marker 235. Therefore, the models can utilize pictures or images 140 of the marker 235 to identify, detect, and track the tip 120. For example, the visual geometry detector 155 can execute at least one model with images 140.

The marker 235 can be applied, attached, connected, coupled, or formed integrally with the tip 120. The marker 235 can be a feature, aspect, or component of the shaft 220 or the tip 120. The marker 235 can be a distinguishing mechanical design, mechanical pattern, or chemical substance of the shaft 220 or the tip 120. The marker 235 can be a fluorescent marking, substance, adhesive material, a bar code, optical code, a quick response (QR) code, or component. The camera 135 can be a fluorescent camera or fluorescent imager. The camera 135 can generate image frames 140 that include data describing fluorescence in the image frames 140, and therefore, the computing system 105 can use the data to track the fluorescent marker 235. Furthermore, the marker 235 can be a distinguishing pattern on the shaft 220 of the instrument 115 that will assist in observing the shaft 220 when it is no covered by a sleeve.

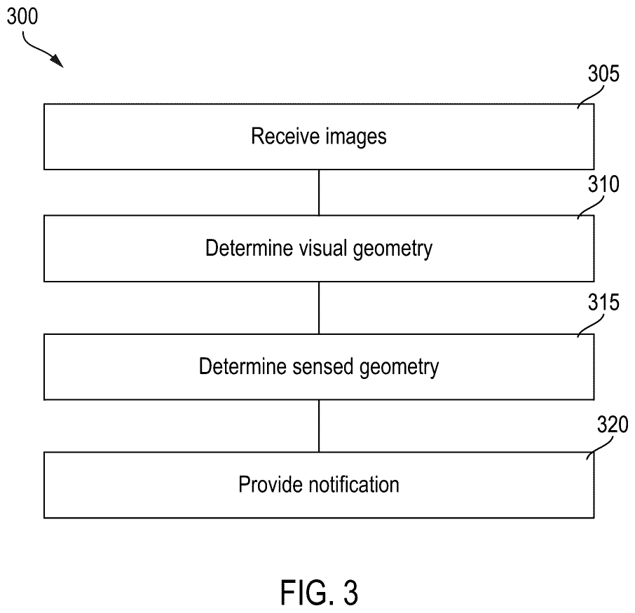

Referring now to FIG. 3, among others, an example method 300 of detecting a state of an instrument 115 of a robotic medical system 100 is shown. At least a portion of the method 300 can be performed by the robotic medical system 100. At least a portion of the method 300 can be performed by the computing system 105. At least a portion of the method 300 can be performed by the obstruction detector 195, the visual geometry detector 155, the sensed geometry detector 170, the pattern matching engine 165, or the user interface manager 183. At least a portion of the method 300 can be performed by the endoscope 127. At least a portion of the method 300 can be performed by the camera 135. At least a portion of the method 300 can be performed by the motor 150 of the endoscope 127. At least a portion of the method 300 can be performed by the instrument 115. At least a portion of the method 300 can be performed by the sensor 130. At least a portion of the method 300 can be performed by the motor 125. At least a portion of the method 300 can be performed by the tip 120. At least a portion of the method 300 can be performed by the user interface device 190.

The method 300 can include an ACT 305 of receiving images. The method 300 can include an ACT 310 of determining a visual geometry. The method 300 can include an ACT 315 of determining a sensed geometry. The method 300 can include an ACT 320 of providing a notification.

At ACT 305, the method 300 can include receiving, by the computing system 105, the images 140. The computing system 105 can receive image frames 140 captured via the camera 135 for a medical session with the robotic medical system 100. The image frames 140 can be captured by the camera 135 and received by the computing system 105 before, after, or during the medical session. The image frames 140 can capture pictures or images of the instrument 115 or the tip 120.

At ACT 310, the method 300 can include determining, by the computing system 105, a visual geometry 160. The method 300 can include executing a visual geometry detector 155 to determine the visual geometry 160. The visual geometry detector 155 can implement a model trained by machine learning, or one or more defined rules or mathematical relations to determine the visual geometry 160 from the image frames 140. The method 300 can include generating or determining the visual geometry 160 by determining a position of the instrument 115 or the tip 120 relative to some point (e.g., the endoscope 127 or the camera 135) or determining an orientation of the tip 120 or the instrument 115 relative to some point (e.g., the endoscope 127 or the camera 135).

At ACT 315, the method 300 can include determining, by the computing system 105, a sensed geometry 180. The method 300 can include receiving data 175 from a sensor 130 of the instrument 115. For example, the method 300 can include receiving data 175 of at least one sensor 130 that describes the operations of at least one motor 125. The sensor data 175 can be current or power data describing current or power sourced by the motors 125. The sensor data 175 can be encoder data that indicates a position of the motor 125. The method 300 can include executing a sensed geometry detector 170 to generate the sensed geometry 180. The method 300 can include determining the sensed geometry 180 with the sensor data 175. The method 300 can include iteratively calculating or determining the sensed geometry 180 and tracking the sensed geometry 180 as new data 175 is received. For example, the method 300 can include maintaining a record of the position or orientation of the instrument 115 or the tip 120, and iteratively update the position or orientation of the instrument 115 or the tip 120 as new data 175 is recorded, e.g., as the motor 125 articulates the tip 120 or moves or reorients the tip 120 via the motor 125.

The sensed geometry detector 170 can determine or track the sensed geometry 180 relative to a point, axis, or plane. For example, the sensed geometry detector 170 and the visual geometry detector 155 can both track the position and orientation of the tip 120 or the instrument 115 relative to the same reference frame, e.g., the same point, axis, or plane. For example, the sensed geometry detector 170 can determine the position and orientation of the tip 120 relative to a position or orientation of the camera 135 of the endoscope 127.

At ACT 320, the method 300 can include providing, by the computing system 105, a notification. The method 300 can include executing a pattern matching engine 165. The method 300 can include comparing the visual geometry 160 with the sensed geometry 180. The method 300 can include comparing a position of the tip 120 determined from the image frames 140 with a position of the tip 120 determined from the data 175. The method 300 can include comparing a orientation of the tip 120 determined from the image frames 140 with an orientation of the tip 120 determined from the data 175. The method 300 can include determining a difference or deviations between the positions or orientations. The greater the different or deviation, the higher the likelihood that the tip 120 is decoupling from the end of the instrument 115, or the greater the likelihood that the tip 120 will decouple from the end of the instrument 115. If the deviation is greater than a threshold, the method 300 can include determining a fault or error state 185. If the deviation is less than the threshold, the method 300 can include determining that a state 185 that indicates that the tip 120 is in a proper or acceptable operating state.

The method 300 can include providing the notification 187 to the user interface device 190 based at least in part on a comparison of the visual geometry 160 with the sensed geometry 180. The notification 187 can be a notification of the state 185 of the instrument 115 or the tip 120 during the medical session. The notification 187 can be displayed within a graphical user interface on the user interface device 190, and provide an indication of the state 185. The notification 187 can be a push notification or a pop-up notification to attract the attention of a doctor, technician, or nurse regarding the state 185 of the tip 120 or the instrument 115.

The method 300 can include receiving an indication that the tip 120 has been installed on the end of the instrument 115. For example, the data 175 can indicate a load on the motor 125 responsive to the tip 120 being installed, and can determine that the tip 120 has been installed using the load on the motor 125. Furthermore, a user can provide input via the user interface device 190 notifying the computing system 105 that the tip 120 has been installed. Responsive to detecting that the tip 120 has been installed, the method 300 can include determining, by the computing system 105, the visual geometry 160 with the image frames 140 of the camera 135, determining the sensed geometry 180 with the data 175, and comparing the visual geometry 160 with the sensed geometry 180.

The method 300 can include detecting, by the computing system 105, that an event has occurred during the medical session. For example, during the medical session, an event can occur, such as the endoscope 127 or the instrument 115 being inserted through the cannula 210. The event can include the instrument 115 making an incision, removing tissue or biological matter, using the tip 120 to perform an action. The event can be detected by analyzing the image frames 140 or receiving user input via the user interface device 190 that indicates the event. Responsive to detecting the event, the method 300 can include determining the visual geometry 160 using the image frames 140. Responsive to detecting the event, the method 300 can include determining the sensed geometry 180 using the data 175. Responsive to detecting the event, the method 300 can include comparing the visual geometry 160 with the sensed geometry 180.

The method 300 can include executing at least one model trained by machine learning to determine the state 185. For example, the method 300 can include training a model with training data. The training data can include data 175 collected from motors 125 from various instruments 115 during various medical sessions, corresponding image frames 140 collected from various cameras 135 of various endoscopes 127 during various medical sessions, and corresponding states 185 (e.g., labeled states) that indicate whether the tip 120 is properly coupled with the instrument 115, improperly coupled with the instrument 115, decoupling or at risk from decoupling from the instrument 115, or has decoupled from the instrument 115. With the trained model, the method 300 can include executing the model with collected image frames 140 and data 175. The method 300 can include executing the model with image frames 140 and data 175 collected at the same or at a corresponding time that the image frames 140 were captured by the camera 135. The model can output the state 185 based on the image frames 140 and the data 175.

In some implementations, the method 300 can include executing a model trained on the visual geometry 160 and the sensed geometry 180. For example, the method 300 can include training a model with machine learning based on a dataset of visual geometries 160, sensed geometries 180, and corresponding states 185. The method 300 can execute the model to compare the visual geometry 160 with the sensed geometry 180 to determine the state 185.

The method 300 can include determining that the tip 120 has detached, decoupled, or fallen off the end of the instrument 115. The method 300 can include detecting, via the image frames 140 and at least one model trained by machine learning. The model can indicate that the tip 120 is not present or is not coupled with the end of the instrument 115. The method 300 can determine that the tip 120 has decoupled from the end of the instrument 115 based on a comparison of the sensed geometry 180 with the visual geometry 160, e.g., a deviation greater than a threshold amount. The method 300 can include recording a time at which the tip 120 decoupled from the end of the instrument 115. For example, responsive to detecting that the tip 120 has decoupled from the end of the instrument 115, the computing system 105 can record and store a timestamp at the time when the tip 120 decoupled form the instrument 115. The method 300 can include selecting or retrieving a set of frames 140 or video clips that occur at or near the timestamp. For example, the method 300 can include selecting frames 140 or video clips a predefined amount of time before the timestamp and a predefined amount of time after the timestamp. The method 300 can include generate, building, or creating a graphical user interface or a notification 187 for a graphical user interface that provides a notification 187 that the tip 120 has decoupled from the instrument 115 and includes the selected frames 140 or video clips. The method 300 can include causing the notification 187 or the graphical user interface to be displayed on the user interface device 190.

In some implementations, the method 300 can include determining a location on a map, such as an anatomical map of the patient, of the tip 120 after the tip 120 decouples from the instrument 115. The method 300 can include determining a trajectory of the tip 120 as the tip 120 falls from the instrument 115. The method 300 can include predicting a location where the tip 120 can be retrieved. The method 300 can predict the location of the tip 120 based on a location of the tip 120 before or as the tip 120 decoupled from the instrument 115 and a movement trajectory of the tip 120 as the tip 120 falls from the instrument 115. The method 300 can include causing a graphical user interface to display the location of the tip 120 on the anatomical map to allow a technician, surgeon, or medical practitioner to retrieve the fallen tip 120.

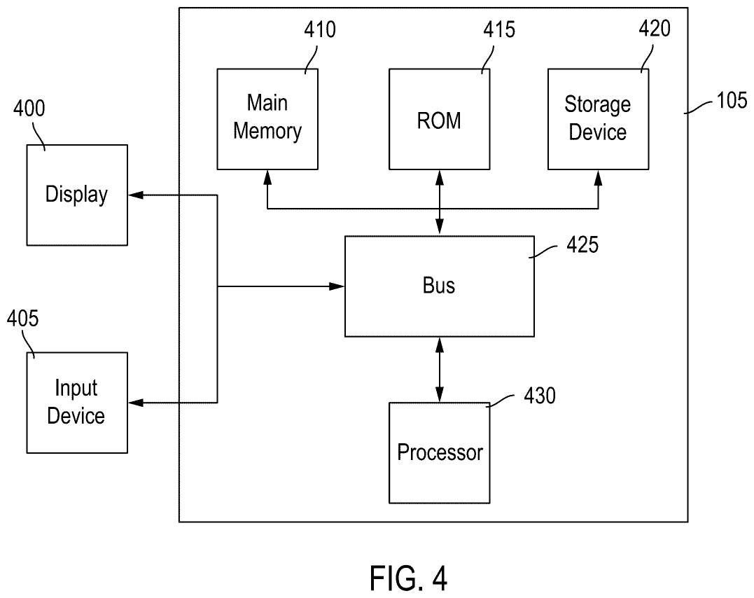

Referring now to FIG. 4, among others, an example block diagram of a computing system 104 is shown. The computing system 105 can include or be used to implement a data processing system or its components. The architecture described in FIG. 4 can be used to implement the computing system 105, the endoscope 127, the instrument 115, the robotic medical system 100. The computing system 105 can include at least one bus 425 or other communication component for communicating information and at least one processor 430 or processing circuit coupled to the bus 425 for processing information. The computing system 105 can include one or more processors 430 or processing circuits coupled to the bus 425 for processing information. The computing system 105 can include at least one main memory 410, such as a random access memory (RAM) or other dynamic storage device, coupled to the bus 425 for storing information, and instructions to be executed by the processor 430. The main memory 410 can be used for storing information during execution of instructions by the processor 430. The computing system 105 can further include at least one read only memory (ROM) 415 or other static storage device coupled to the bus 425 for storing static information and instructions for the processor 430. A storage device 420, such as a solid state device, magnetic disk or optical disk, can be coupled to the bus 425 to persistently store information and instructions.

The computing system 105 can be coupled via the bus 425 to a display 400, such as a liquid crystal display, or active matrix display. The display 400 can display information to a user. An input device 405, such as a keyboard or voice interface can be coupled to the bus 425 for communicating information and commands to the processor 430. The input device 405 can include a touch screen of the display 400. The input device 405 can include a cursor control, such as a mouse, a trackball, or cursor direction keys, for communicating direction information and command selections to the processor 430 and for controlling cursor movement on the display 400.

The processes, systems and methods described herein can be implemented by the computing system 105 in response to the processor 430 executing an arrangement of instructions contained in main memory 410. Such instructions can be read into main memory 410 from another computer-readable medium, such as the storage device 420. Execution of the arrangement of instructions contained in main memory 410 causes the computing system 105 to perform the illustrative processes described herein. One or more processors in a multi-processing arrangement can be employed to execute the instructions contained in main memory 410. Hard-wired circuitry can be used in place of or in combination with software instructions together with the systems and methods described herein. Systems and methods described herein are not limited to any specific combination of hardware circuitry and software.

Although an example computing system has been described in FIG. 4, the subject matter including the operations described in this specification can be implemented in other types of digital electronic circuitry, or in computer software, firmware, or hardware, including the structures disclosed in this specification and their structural equivalents, or in combinations of one or more of them.

Some of the description herein emphasizes the structural independence of the aspects of the system components or groupings of operations and responsibilities of these system components. Other groupings that execute similar overall operations are within the scope of the present application. Modules can be implemented in hardware or as computer instructions on a non-transient computer readable storage medium, and modules can be distributed across various hardware or computer based components.

The systems described above can provide multiple ones of any or each of those components and these components can be provided on either a standalone system or on multiple instantiations in a distributed system. In addition, the systems and methods described above can be provided as one or more computer-readable programs or executable instructions embodied on or in one or more articles of manufacture. The article of manufacture can be cloud storage, a hard disk, a CD-ROM, a flash memory card, a PROM, a RAM, a ROM, or a magnetic tape. In general, the computer-readable programs can be implemented in any programming language, such as LISP, PERL, C, C++, C#, PROLOG, Python, or in any byte code language such as JAVA. The software programs or executable instructions can be stored on or in one or more articles of manufacture as object code.

Example and non-limiting module implementation elements include sensors providing any value determined herein, sensors providing any value that is a precursor to a value determined herein, datalink or network hardware including communication chips, oscillating crystals, communication links, cables, twisted pair wiring, coaxial wiring, shielded wiring, transmitters, receivers, or transceivers, logic circuits, hard-wired logic circuits, reconfigurable logic circuits in a particular non-transient state configured according to the module specification, any actuator including at least an electrical, hydraulic, or pneumatic actuator, a solenoid, an op-amp, analog control elements (springs, filters, integrators, adders, dividers, gain elements), or digital control elements.

The subject matter and the operations described in this specification can be implemented in digital electronic circuitry, or in computer software, firmware, or hardware, including the structures disclosed in this specification and their structural equivalents, or in combinations of one or more of them. The subject matter described in this specification can be implemented as one or more computer programs, e.g., one or more circuits of computer program instructions, encoded on one or more computer storage media for execution by, or to control the operation of, data processing apparatuses. Alternatively or in addition, the program instructions can be encoded on an artificially generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. While a computer storage medium is not a propagated signal, a computer storage medium can be a source or destination of computer program instructions encoded in an artificially generated propagated signal. The computer storage medium can also be, or be included in, one or more separate components or media (e.g., multiple CDs, disks, or other storage devices including cloud storage). The operations described in this specification can be implemented as operations performed by a data processing apparatus on data stored on one or more computer-readable storage devices or received from other sources.

The terms “computing device”, “component” or “data processing apparatus” or the like encompass various apparatuses, devices, and machines for processing data, including by way of example a programmable processor, a computer, a system on a chip, or multiple ones, or combinations of the foregoing. The apparatus can include special purpose logic circuitry, e.g., an FPGA (field programmable gate array) or an ASIC (application specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, app, script, or code) can be written in any form of programming language, including compiled or interpreted languages, declarative or procedural languages, and can be deployed in any form, including as a stand-alone program or as a module, component, subroutine, object, or other unit suitable for use in a computing environment. A computer program can correspond to a file in a file system. A computer program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatuses can also be implemented as, special purpose logic circuitry, e.g., an FPGA (field programmable gate array) or an ASIC (application specific integrated circuit). Devices suitable for storing computer program instructions and data can include non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

The subject matter described herein can be implemented in a computing system that includes a back end component, e.g., as a data server, or that includes a middleware component, e.g., an application server, or that includes a front end component, e.g., a client computer having a graphical user interface or a web browser through which a user can interact with an implementation of the subject matter described in this specification, or a combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

While operations are depicted in the drawings in a particular order, such operations are not required to be performed in the particular order shown or in sequential order, and all illustrated operations are not required to be performed. Actions described herein can be performed in a different order.

Having now described some illustrative implementations, it is apparent that the foregoing is illustrative and not limiting, having been presented by way of example. In particular, although many of the examples presented herein involve specific combinations of method acts or system elements, those acts and those elements may be combined in other ways to accomplish the same objectives. ACTs, elements and features discussed in connection with one implementation are not intended to be excluded from a similar role in other implementations.

The phraseology and terminology used herein is for the purpose of description and should not be regarded as limiting. The use of “including” “comprising” “having” “containing” “involving” “characterized by” “characterized in that” and variations thereof herein, is meant to encompass the items listed thereafter, equivalents thereof, and additional items, as well as alternate implementations consisting of the items listed thereafter exclusively. In one implementation, the systems and methods described herein consist of one, each combination of more than one, or all of the described elements, acts, or components.

Any references to implementations or elements or acts of the systems and methods herein referred to in the singular may also embrace implementations including a plurality of these elements, and any references in plural to any implementation or element or act herein may also embrace implementations including only a single element. References in the singular or plural form are not intended to limit the presently disclosed systems or methods, their components, acts, or elements to single or plural configurations. References to any ACT or element being based on any information, act or element may include implementations where the act or element is based at least in part on any information, act, or element.

Any implementation disclosed herein may be combined with any other implementation or example, and references to “an implementation,” “some implementations,” “one implementation” or the like are not necessarily mutually exclusive and are intended to indicate that a particular feature, structure, or characteristic described in connection with the implementation may be included in at least one implementation or example. Such terms as used herein are not necessarily all referring to the same implementation. Any implementation may be combined with any other implementation, inclusively or exclusively, in any manner consistent with the aspects and implementations disclosed herein.

References to “or” may be construed as inclusive so that any terms described using “or” may indicate any of a single, more than one, and all of the described terms. References to at least one of a conjunctive list of terms may be construed as an inclusive OR to indicate any of a single, more than one, and all of the described terms. For example, a reference to “at least one of ‘A’ and ‘B’” can include only ‘A’, only ‘B’, as well as both ‘A’ and ‘B’. Such references used in conjunction with “comprising” or other open terminology can include additional items.

Where technical features in the drawings, detailed description or any claim are followed by reference signs, the reference signs have been included to increase the intelligibility of the drawings, detailed description, and claims. Accordingly, neither the reference signs nor their absence have any limiting effect on the scope of any claim elements.

Modifications of described elements and acts such as variations in sizes, dimensions, structures, shapes and proportions of the various elements, values of parameters, mounting arrangements, use of materials, colors, orientations can occur without materially departing from the teachings and advantages of the subject matter disclosed herein. For example, elements shown as integrally formed can be constructed of multiple parts or elements, the position of elements can be reversed or otherwise varied, and the nature or number of discrete elements or positions can be altered or varied. Other substitutions, modifications, changes and omissions can also be made in the design, operating conditions and arrangement of the disclosed elements and operations without departing from the scope of the present disclosure.

What is claimed is:

1. A system, comprising:
one or more processors, coupled with memory, to:
receive one or more image frames captured via a camera for a medical session with a robotic medical system;
determine, based at least in part on the one or more image frames, a visual geometry of an instrument configured to perform a procedure via the robotic medical system during at least a portion of the medical session;
determine, based at least in part on data received from a sensor of a motor of the robotic medical system, a sensed geometry of the instrument; and
provide, based at least in part on a comparison of the visual geometry with the sensed geometry, a notification of a state of the instrument during the medical session.

2. The system of claim 1, wherein the one or more processors are further configured to:
operate a motor of a stereoscope comprising the camera to pass through a cannula;
operate the motor of the stereoscope to move the camera away from a longitudinal axis of the cannula by an amount;
detect that the camera is moved away from the longitudinal axis of the cannula by the amount; and
operate at least one motor of an arm comprising the instrument to pass through the cannula responsive to a detection that that the camera is moved away from the longitudinal axis of the cannula by the amount.

3. The system of claim 1, wherein the one or more processors are further configured to:
receive an indication that the instrument is installed on an arm of the robotic medical system; and
compare the visual geometry with the sensed geometry to determine the state of the instrument responsive to the indication that the instrument is installed on the arm of the robotic medical system.

4. The system of claim 1, wherein the one or more processors are further configured to:
detect that an event of the medical session occurred;
determine the visual geometry responsive to a detection of the event;
determine the sensed geometry responsive to the detection of the event; and
compare the visual geometry with the sensed geometry to determine the state responsive to the detection of the event.

5. The system of claim 1, wherein the one or more processors are further configured to:
determine a position or orientation of the camera of an endoscope of the robotic medical system;
determine the visual geometry of the instrument relative to the position or the orientation of the endoscope; and
determine the sensed geometry of the instrument relative to the position or the orientation of the camera on the endoscope.

6. The system of claim 1, wherein the one or more processors are further configured to:
detect, based at least in part on the one or more image frames, an object blocking a view of the instrument; and
suppress the notification of an error state of the instrument responsive to a detection of the object blocking the view of the instrument.

7. The system of claim 1, wherein the one or more processors are further configured to:
detect, based at least in part on the one or more image frames, a pattern of a sleeve covering at least a portion of an arm of the robotic medical system comprising the camera; and
generate the notification of the state of the instrument based on the pattern of the sleeve.

8. The system of claim 1, wherein the one or more processors are further configured to:
detect, based at least in part on the one or more image frames, a marker of the instrument, the marker applied to the instrument; and
track, based on the one or more image frames, the marker to determine the visual geometry of the instrument.

9. The system of claim 1, wherein the one or more processors are further configured to:
periodically or continuously determine the visual geometry, determine the sensed geometry, and provide the notification of the state of the instrument during the medical session.

10. The system of claim 1, wherein the one or more processors are further configured to:
execute a model trained on machine learning based on the data from the sensor of the motor and the one or more image frames to determine the state of the instrument; and
provide the notification of the state of the instrument based on execution of the model.

11. The system of claim 1, wherein the one or more processors are further configured to:
determine, based at least in part on the comparison of the visual geometry with the sensed geometry, that the instrument decoupled from an arm of the robotic medical system;
determine a time at which the instrument decoupled from the arm of the robotic medical system;
select at least one frame of a plurality of frames captured by the camera based on the time at which the instrument decoupled from the arm; and
provide a graphical user interface comprising an indication that the instrument decoupled from the arm and the selected frame.

12. A method, comprising:
receiving, by one or more processors coupled with memory, one or more image frames captured via a camera for a medical session with a robotic medical system;
determining, by the one or more processors, based at least in part on the one or more image frames, a visual geometry of an instrument configured to perform a procedure via the robotic medical system during at least a portion of the medical session;
determining, by the one or more processors, based at least in part on data received from a sensor of a motor of the robotic medical system, a sensed geometry of the instrument; and
providing, by the one or more processors, based at least in part on a comparison of the visual geometry with the sensed geometry, a notification of a state of the instrument during the medical session.

13. The method of claim 12, comprising:
operating, by the one or more processors, a motor of a stereoscope of the comprising the camera to pass through a cannula;
operating, by the one or more processors, the motor of the stereoscope to move the camera away from a longitudinal axis of the cannula by an amount;
detecting, by the one or more processors, that the camera is moved away from the longitudinal axis of the cannula by the amount; and
operating, by the one or more processors, at least one motor of an arm comprising the instrument to pass through the cannula responsive to a detection that that the camera is moved away from the longitudinal axis of the cannula by the amount.

14. The method of claim 12, comprising:
receiving, by the one or more processors, an indication that the instrument is installed on an arm of the robotic medical system; and
comparing, by the one or more processors, the visual geometry with the sensed geometry to determine the state of the instrument responsive to the indication that the instrument is installed on the arm of the robotic medical system.

15. The method of claim 12, comprising:
detecting, by the one or more processors, that an event of the medical session occurred;
determining, by the one or more processors, the visual geometry responsive to a detection of the event;
determining, by the one or more processors, the sensed geometry responsive to the detection of the event; and
comparing, by the one or more processors, the visual geometry with the sensed geometry to determine the state responsive to the detection of the event.

16. The method of claim 12, comprising:
determining, by the one or more processors, a position or orientation of the camera of an endoscope of the robotic medical system;
determining, by the one or more processors, the visual geometry of the instrument relative to the position or the orientation of the endoscope; and
determining, by the one or more processors, the sensed geometry of the instrument relative to the position or the orientation of the camera on the endoscope.

17. The method of claim 12, comprising:
executing, by the one or more processors, a model trained on machine learning based on the data from the sensor of the motor and the one or more image frames to determine the state of the instrument; and
providing, by the one or more processors, the notification of the state of the instrument based on execution of the model.

18. The method of claim 12, comprising:
determining, by the one or more processors, based at least in part on the comparison of the visual geometry with the sensed geometry, that the instrument decoupled from an arm of the robotic medical system;
determining, by the one or more processors, a time at which the instrument decoupled from the arm of the robotic medical system;
selecting, by the one or more processors, at least one frame of a plurality of frames captured by the camera based on the time at which the instrument decoupled from the arm; and
providing, by the one or more processors, a graphical user interface comprising an indication that the instrument decoupled from the arm and the selected frame.

19. One or more storage media storing instructions thereon, that, when executed by one or more processors, cause the one or more processors to:
receive one or more image frames captured via a camera for a manufacturing session for an instrument;
determine, based at least in part on the one or more image frames, a visual geometry of the instrument manufactured to perform a medical procedure via a robotic medical system;
determine, based at least in part on data received from a sensor of a motor, a sensed geometry of the instrument; and
provide, based at least in part on a comparison of the visual geometry with the sensed geometry, a notification of a state of the instrument.

20. The one or more storage media of claim 19, wherein the instructions cause the one or more processors to:
determine, based at least in part on the comparison of the visual geometry with the sensed geometry, that the instrument decoupled from an arm of the robotic medical system;
determine a time at which the instrument decoupled from the arm of the robotic medical system;
select at least one frame of a plurality of frames captured by the camera based on the time at which the instrument decoupled from the arm; and
provide a graphical user interface comprising an indication that the instrument decoupled from the arm and the selected frame.
